(19) United States
(12) Patent Application Publication
(10) Pub. No.: US 2007/0239208 A1
(43) Pub. Date: Oct. 11, 2007

(54) SURGICAL IMPLANTATION DEVICE AND METHOD

(76) Inventor: Bruce S. Crawford, Reno, NV (US)

Correspondence Address: SIERRA PATENT GROUP, LTD., 1657 Hwy 395, Suite 202, Minden, NV, 89423, US

(21) Appl. No.: 11/670,195

(22) Filed: Feb. 1, 2007

Related U.S. Application Data
(60) Provisional application No. 60/809,877, filed on Jun. 1, 2006.
(60) Provisional application No. 60/789,845, filed on Apr. 5, 2006.

(51) Int. Cl. A61B 17/04

(52) U.S. Cl. 606/232

(57) ABSTRACT

A surgical implantation device comprising: a flexible conduit having a proximal end and a distal end; a means for securing the flexible conduit to an operator's hand over a surgical glove such that the position of the conduit may be adjusted by the movement of one of the operator's fingers; a tissue anchor configured to enter the opening in the proximal end, be advanced through the conduit, and exit the opening in the distal end where it can be deployed beyond the surface of a target tissue layer; a suture element connected to the tissue anchor, the suture element configured to extend from the tissue anchor through the conduit; and a flexible rod having a proximal end and a distal end, the distal end of the rod configured to advance the tissue anchor through the conduit and deploy the tissue anchor beyond the surface of the target tissue layer.

### CROSS-REFERENCE TO RELATED APPLICATIONS

This application claims priority to U.S. Provisional Patent Application Ser. No. 60/809,877, filed Jun. 1, 2006, and to U.S. Provisional Patent Application Ser. No. 60/789,845, filed Apr. 5, 2006, both of which are hereby incorporated by reference as if set forth herein.

### BACKGROUND

1. Field of the Disclosure

The present invention relates to a surgical introducer apparatus and method for inserting material to be retained within a living body.

2. Background

Securing materials such as graft material and suture material into the body is often used in the context of therapeutic surgical procedures. One area in which such procedures are often performed is vaginal reconstructive surgery. In this field, graft material is used to repair pelvic support defects such as cystoceles, rectoceles, and vaginal vault prolapse. The accurate placement of graft material or suture material at various positions via small incisions is desirable and has been an area of great progress in recent years. The “open access” Capio® (Boston Scientific, Natick, Mass.) device is commonly used to place suture material through the fibromuscular layers of the pelvic floor in the context of vaginal reconstructive surgery. This instrument passes a bullet-tipped suture through the layers of the pelvic floor and transfers the bullet-tipped suture to a receiving end, effectively placing a single stitch without the necessity of visualization. Limitations of this device include the following.

First, the exit point of the suture is about 1 cm from the entrance point, effectively displacing the point of fixation of the graft material more distally than desired. In addition, because a loop of suture is created using the Capio® device, the possibility of inadvertently incorporating a portion of the bladder wall and/or the ureter exists. If this occurs, the bladder wall and/or the ureter may be constricted, obstructed, or otherwise injured as the suture is tied down.

Second, use of this device in the repair of vaginal prolapse requires the operator to insert the device and his own finger into the paravaginal dissection. This insertion requires a dissection large enough to accommodate these two separate structures. Furthermore, feeling where the tip of the Capio® is located can be challenging because it is a thick and rigid instrument.

Third, the Capio® has a limited depth of penetration (estimated 3 mm). Especially when placed against a flat surface such as the pelvic floor musculature. Depth of penetration is a critical factor in obtaining a secure purchase of tissue. A secure purchase of tissue is essential in creating an adequate and durable repair.

A substantial improvement to the present state of the art would be afforded by an instrument that allows these same points of attachment to be obtained without the above-described deficiencies.

Several other devices have been introduced recently to facilitate placement of graft material at various positions within the pelvis for the purpose of vaginal reconstructive surgery. The IVS Tunneler™ (Tyco Corp., Princeton, N.J.) was FDA approved as a method of obtaining a secure fixation point in the pelvic floor musculature in the context of vaginal vault suspension. This device involves the insertion of an introducer via a peri-anal incision through the ishiorectal fossa to the posterior surface of the coccygeus muscle. The introducer is then passed through the coccygeus muscle at which point a graft is loaded onto the tip of the introducer. The introducer is then withdrawn and the graft is pulled through the vaginal incision and out through the perianal incision. This procedure is then repeated on the contralateral side. The midline of the graft is sutured to the vaginal vault. Since the introduction of this instrument, several companies have introduced similar technologies that provide a method of creating secure points of attachment for either vaginal fibromuscularis or graft material. Some examples of these so-called trochar systems include: Avaulta™ (CR Bard, Inc., Murray Hill, N.J.), Apogee/Perigee™ (American Medical Systems, Minnetonka, Mich.). Deficiencies with these products include the following.

These products involve the blind passage of introducers for relatively long distances along the posterior surface of the pelvic floor, where important nerves and vascular structures may be injured.

The use of these products involves the necessity to leave long tags of permanent synthetic graft material between the posterior surface of the pelvic floor and the groin or perianal incision sites. A greater amount of foreign body may confer a greater risk of infection and or erosion.

The technical difficulty in passing the introducers of these products to the desired position along the pelvic floor translates into greater surgical risk, especially for new adopters of the technology.

These products result in the need for additional surgical incisions outside the vagina.

A substantial improvement to the present state of the art would be afforded by an instrument that allows these same points of attachment to be obtained without the above-described deficiencies.

Levy et al., in U.S. Pat. Nos. 6,332,888 and 6,475,135, describe a finger-guided surgical instrument with applications in the treatment of female urinary incontinence and vaginal prolapse. The instrument is a “thimble-like element” that fits over the operators finger in such a way that the tactile sensory function of the finger is relatively preserved. The design contains a mechanism to pass a surgical needle through and stitch tissue that the operator's finger abuts. Levy et al. describe an instrument in which there are channels within the housing of the “thimble-like element” to allow for passage of a surgical needle, an anchor guide and anchor, a capillary tube, or other instrument into tissue in close juxtaposition to the operator's finger tip. Deficiencies with this instrument include: (1) Relatively bulky design that is mechanically complex; (2) Position of the guide for the anchor element is lateral to the operator's finger tip and, therefore, not at the exact anatomic site palpated by the operators finger tip; (3) Relatively cumbersome in terms of reloading the instrument after placement of a suture or tissue anchor; (4) Relatively expensive to produce; and (5) In the embodiment that describes wearing the device over the ventral surface of the operator's finger, the insertion instruments extend from the distal tip of the instrument rather than immediately under the ventral pad of the operators finger. The rigid and bulky nature of this instrument would interfere with the operator's ability to discriminate landmarks as the device is deployed.

A substantial improvement to the present state of the art would be afforded by an instrument that allows for the tactile-guided placement of a tissue anchor without the above described deficiencies.

Various devices and techniques have been described to place tissue anchors within the soft tissues of the human body. The vast majority of these devices describe anchors that are intended to be placed within the substance of a muscle or tendon. These types of anchors often utilize barbs, spines, or other designs, such as a screw or helix shape, that are intended to grab the surrounding tissues so as to prevent the anchor from being dislodged. These types of anchors are most suitable for bulky, thick, target tissues, as the pull out force will be proportionate to the strength of the host tissue between the anchor and the point of insertion. These types of anchors are referred to as “partial thickness” anchors.

For very thin muscles, partial thickness anchors are suboptimal because only a narrow band of tissue will remain between the insertion site and the anchor. When working with very thin muscles, such as the pelvic floor muscles, it would be ideal to utilize the strength of the full thickness of the target tissue. This utilization could be accomplished by placing the anchor through the target tissue such that it comes to rest against the deep surface of the target tissue. Various bar-shaped or T-shaped anchors have been described that could be used in this fashion. In the context of vaginal reconstructive surgery, it would be suboptimal to have any portion of the anchor impinging on the vaginal lumen. If a T-shaped anchor were used for this purpose, the vertical member of the T could cause male dysparunia or erosion through the vaginal epithelium. Because the pelvic floor muscles are very thin, even a bar-type anchor may be palpable through the vaginal epithelium.

Furthermore, the solid bar-type anchors have several drawbacks. This anchor-type involves placement of a relatively large foreign body, which requires a relatively large insertion tract, and may be associated with greater risk of erosion into surrounding tissues and infection.

A substantial improvement to the present state of the art would be afforded by an instrument that allows full thickness anchor placement without the above-described deficiencies.

### SUMMARY

The present invention provides a new, useful and non-obvious finger-directed implant system, device and method for precisely deploying a tissue anchor within the soft tissues of a living body via a minimally invasive technique. The system comprises four basic elements: (1) a conduit or conduit-glove assembly; (2) an insertion rod; (3) a suture element; and (4) a tissue anchor.

According to one aspect of the present invention, there is provided a glove-like garment that contains a conduit along the ventral surface of the index finger that admits a flexible rod having a distal tip designed to penetrate the target tissue. The distal end of the rod is fashioned to carry the tissue anchor element into the target tissue and deposit the anchor element within the target tissue as the rod is withdrawn. The channeled glove contains a window over the tip of the guiding finger, centrally aligned with the tip of the conduit, allowing the operator to palpate the position of anatomical landmarks before the implantable element is deployed. Once the tip of the operator's finger is in position over the desired fixation point, the implantable element can be deployed by advancing the rod through the channel in the glove (i.e., the conduit). As the rod is advanced, it penetrates the host tissue at a point immediately beneath, and centrally aligned with, the ventral surface of the distal phalanx of the operator's finger. As the rod is advanced further, the implantable element is moved into the host tissue. The rod is advanced until the implantable element has moved through the host tissue layer. Once deposited beyond the deep surface of the target tissue layer, the rod is withdrawn from the channel, leaving the implantable element and attached sutures in place.

As an alternative to the glove-conduit assembly, a conduit may be produced as a separate device, along with a means of attaching it to a standard surgical glove. For example, a conduit with an adhesive backing along one side could be used to attach it to any surgical glove. This configuration would solve the problem of trying to produce a single glove garment that would satisfactorily fit all operators. This solo conduit may be positioned along any finger the operator chooses to use as the guiding finger. The solo conduit would likely be easier to affix to the operator's hand than a separate glove device. In addition, a solo conduit could be produced less expensively and without the issue of right-left laterality that would exist with a glove-conduit assembly.

In one embodiment, the implantable tissue anchor is a compressible ring-like element with memory of its natural annular, or semi-annular, shape. Covering the central area of the ring is a biocompatible fabric. The fabric contains a window at its distal end that allows the ring to be mounted onto the tip of the insertion rod. Suture material is attached to the tissue anchor such that it can be used, after the ring is deployed, for surgical purposes including, but not limited to, the fixation of graft material to the site where the anchor is deployed.

In one version, the suture material is attached to the central region of the fabric that covers the central area of the ring. In a preferred embodiment, the frame of the tissue anchor includes a barb on the convex surface of the proximal edge designed to snare the host tissue and dislodge the implant from the insertion rod as the rod is withdrawn. The thickness of the frame is such that it is compatible with a recessed portion of the introduction rod. As the insertion rod and implantable element are introduced into the glove conduit the frame becomes compressed and elongated. The rim of the tissue anchor fits into the lateral and dorsal recesses of the insertion rod. The biocompatible fabric that covers the central portion of the frame drapes over the dorsal aspect of the insertion rod. The suture material attached to the central portion of the fabric passes along the side of the insertion rod as it is inserted into the conduit.

In a preferred embodiment, the surgical implantation device comprises a flexible conduit having a proximal end and a distal end opposite the proximal end. The flexible conduit comprises an opening at the proximal end in communication with an opening at the distal end. The device also comprises a means for securing the flexible conduit to an operator's hand over a surgical glove such that the position of the conduit may be adjusted by the movement of one of the operator's fingers. The device further comprises a tissue anchor configured to enter the opening in the proximal end, be advanced through the conduit, and exit the opening in the distal end, where it can be deployed beyond the surface of a target tissue layer. The device also comprises a suture element connected to the tissue anchor. The suture element is configured to extend from the tissue anchor through the conduit, from the distal end to the proximal end. A flexible rod is also included, having a proximal end and a distal end opposite the proximal end. The distal end of the rod is configured to advance the tissue anchor through the conduit from the opening in the proximal end of the conduit to the opening in the distal end of the conduit and deploy the tissue anchor beyond the surface of the target tissue layer.

### BRIEF DESCRIPTION OF THE DRAWINGS

FIG. 1 is a ventral view of an exemplary conduit-glove assembly with a conduit extending across the palm region, along the ventral surface of the operator's index finger, to the distal phalanx of the index finger in accordance with the present invention;

FIG. 2 is lateral view of an exemplary insertion rod in accordance with the present invention;

FIG. 3 is a plan view of an exemplary insertion rod in accordance with the present invention;

FIG. 4 is a plan view of an exemplary tissue anchor in accordance with the present invention;

FIG. 5 is a lateral view of an exemplary embodiment of the insertion rod/tissue anchor assembly in accordance with the present invention;

FIG. 6 is a ventral view of an exemplary embodiment of the tissue anchor-insertion rod assembly being advanced along the glove conduit in accordance with the present invention;

FIG. 7 is a ventral view of an exemplary embodiment of the tissue anchor-insertion rod assembly emerging from the distal end of the glove conduit in accordance with the present invention;

FIG. 8A is a ventral view of an exemplary embodiment of the distal end of the glove conduit abutting the target tissue and the tissue anchor-insertion rod assembly advancing through the target tissue layer in accordance with the present invention;

FIG. 8B is an enlarged view of the anchor-insertion rod assembly, target tissue layer, and finger tip shown in FIG. 8A;

FIG. 9A is a ventral view of an exemplary embodiment of the rod being withdrawn from the target tissue, leaving the anchor on the deep side of the target tissue layer, in accordance with the present invention;

FIG. 9B is an enlarged view of the anchor, target tissue layer, and finger tip shown in FIG. 9A; and

FIG. 10 illustrates an exemplary embodiment of the tissue anchor deposited within the target tissue in accordance with the present invention.

### DETAILED DESCRIPTION

Persons of ordinary skill in the art will realize that the following description is illustrative only and not in any way limiting. Other modifications and improvements will readily suggest themselves to such skilled persons having the benefit of this disclosure. In the following description, like reference numerals refer to like elements throughout.

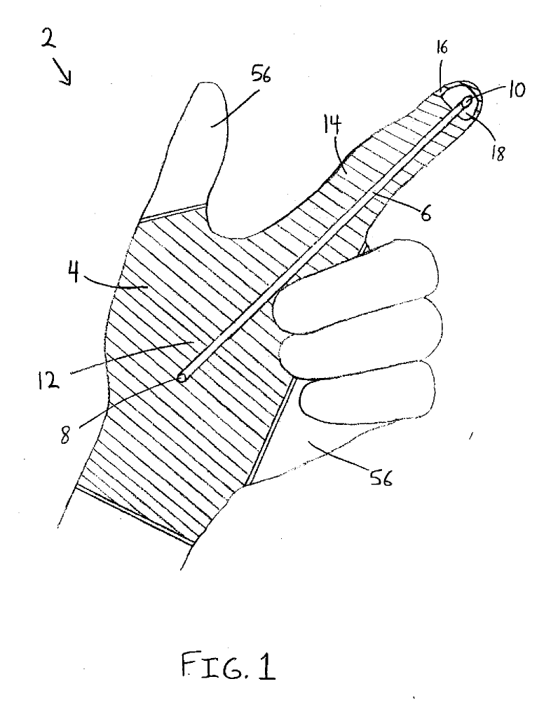

The present invention provides a tactile-guided system 2 for the precise, and minimally invasive introduction of an instrument into a body. The system 2 can be applied to surgical procedures involving placement of tissue anchors, incision and drainage of cystic structures, radiological localization of an anatomic structure, and injection of materials into the tissues of a body. The functionality of this instrument will be described with reference to FIGS. 1-10.

FIG. 1 illustrates a glove garment 4 with an attached conduit 6 extending from the palm region 12 of the ventral surface of the operators hand, along the ventral surface of the operator's index finger 14, to the operator's distal phalanx 16. A window 12 may be disposed in the glove fabric over the ventral surface of the operator's distal phalanx 16 such that it exposes a portion of the operator's underglove (standard surgical glove) 56. This window 18 allows the operator optimal tactile discrimination of anatomic landmarks relevant to the surgical procedure being performed.

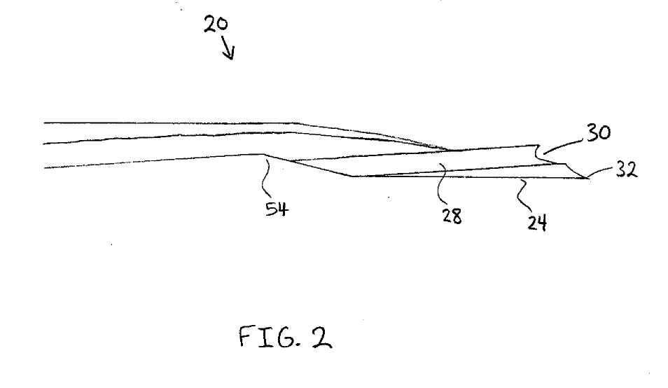

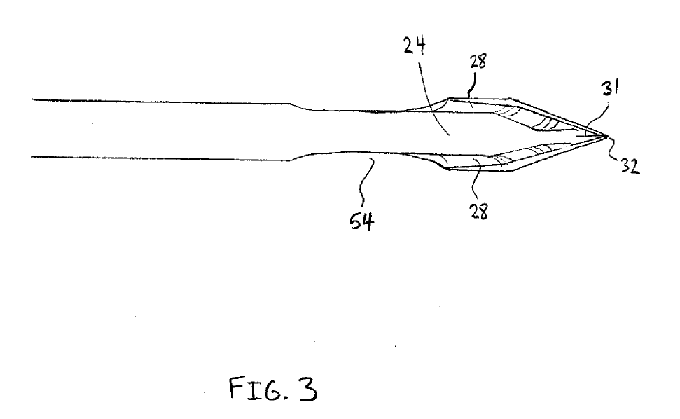

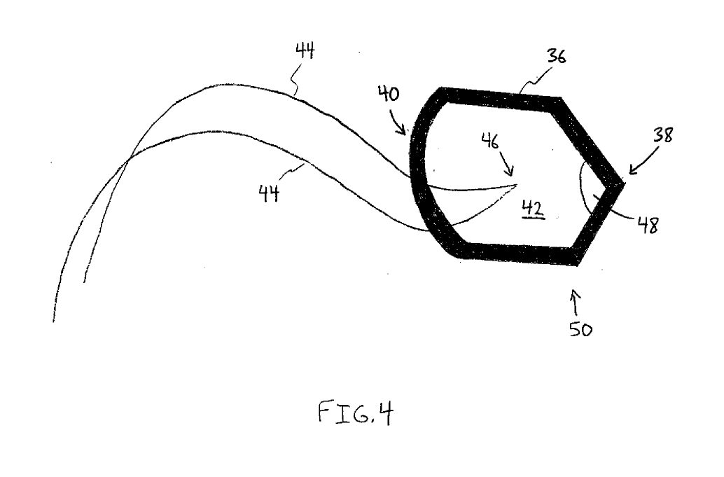

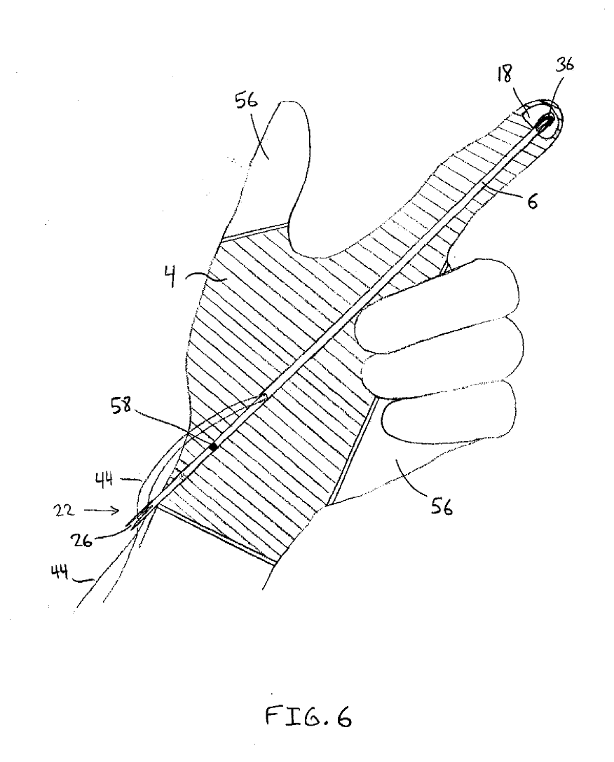

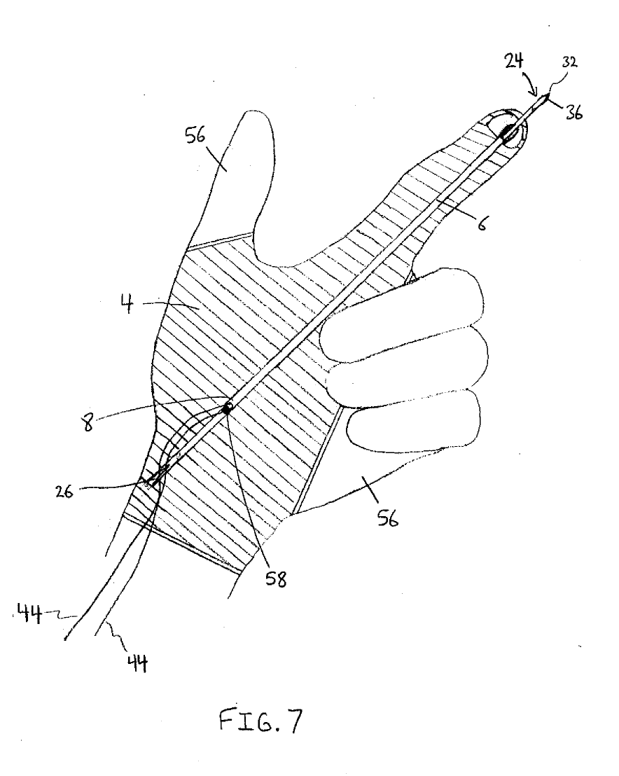

The conduit 6 contains a first opening at its proximal end 8 and second opening at its distal end 10 in order to allow passage of an insertion rod, such as insertion rod 20 shown in FIGS. 2-3, and a tissue anchor, such as tissue anchor 50 shown in FIG. 4, through the conduit 6, as seen in FIGS. 6-7. In a preferred embodiment, proximal end 8 is substantially blunt, while distal end 10 is substantially tapered, preferably forming a sharp edge. However, it is contemplated that proximal end 8 and distal end 10 may be formed in a variety of different shapes.

FIGS. 1 and 6-9B show glove garment 4 covering a majority of palm region and completely envelopes the index finger, with the exception of window 18 on the ventral surface of the distal phalanx 16, while leaving the underglove 56 exposed on a majority of the thumb, the ring finger and the little finger. In another embodiment, glove garment 4 may covers the operator's thumb in addition to the palm region and the index finger. It is contemplated that different finger configurations of glove garment 4 are well within the scope of the present invention. Preferably, glove garment 4 is configured to be worn over the operator's hand such that it covers at least a portion of the operator's palm and wraps around the back of the operator's hand to securely hold itself in position on the operator's hand. Additionally, glove garment 4 is preferably configured to extend over a substantial portion of at least one of the index finger and the middle finger.

Glove garment 4 provides a secure, yet easily removable, surface for holding conduit 6 in position on the operator's hand during use. The conduit element 6 may extend along the ventral surface of the operator's index finger 14 to a window region 18 in the glove element 4 over the ventral surface of the distal phalanx 16 of the operator's index finger. In a preferred embodiment, conduit 6 is configured to extend all the way from the operator's palm region 12 to the distal phalanx 16 of the operator's index finger.

It has been appreciated that the conduit element 6 may be provided separate from any glove garment. In this alternative embodiment, conduit 6 is provided along with a means of securing it to a standard surgical glove. One method of securing such a solo conduit would be to cover one surface of the conduit 6 with an adhesive tape having a removable backing such that when the backing is removed, the tape extends on either side of the conduit 6, thereby allowing the conduit 6 to be secured to a standard surgical glove at a position desired by the operator. Other securing means may be used in addition, or as an alternative, to an adhesive. This simplification of the present invention eliminates the need for multiple sizes of glove garments to accommodate operators with different sized hands. In addition, the solo conduit would be less expensive to produce, package, and ship, than a glove-conduit assembly. Production of a solo conduit eliminates the issue of right-left laterality inherent to the glove-conduit assembly.

In a preferred embodiment, conduit element 6 is made of a flexible material such that it will conform to the contour of the ventral surface of the operator's palm and finger as the operator's finger is flexed to the degree necessary to palpate the site desired for anchor placement.

In a preferred embodiment, insertion rod 20 is an elongated, flexible instrument having a proximal end 22 and a distal end 24 opposite proximal end 22. Insertion rod 20 is configured to fit within the conduit element 6. The proximal end 22 is preferably blunt, so as to avoid any accidental incisions, and may comprise a slit 26 that is configured to receive the suture elements 44 of the tissue anchor 50, shown in FIG. 4, in such a way that the suture elements 44 are held securely in place within slit 26, as seen in FIG. 6, while the rod 20 is inserted through the conduit 6 and into host tissue. By securing the suture elements 44 to the proximal end 22 of the insertion rod 20 in this way, and under a reasonable amount of tension, the anchor 50 will be held in a stable position on the distal end 24 of the insertion rod 20. This secure positioning will be useful when inserting the insertion rod/anchor assembly into the proximal end 8 of the conduit 6, and will help prevent premature deployment of the anchor 50.

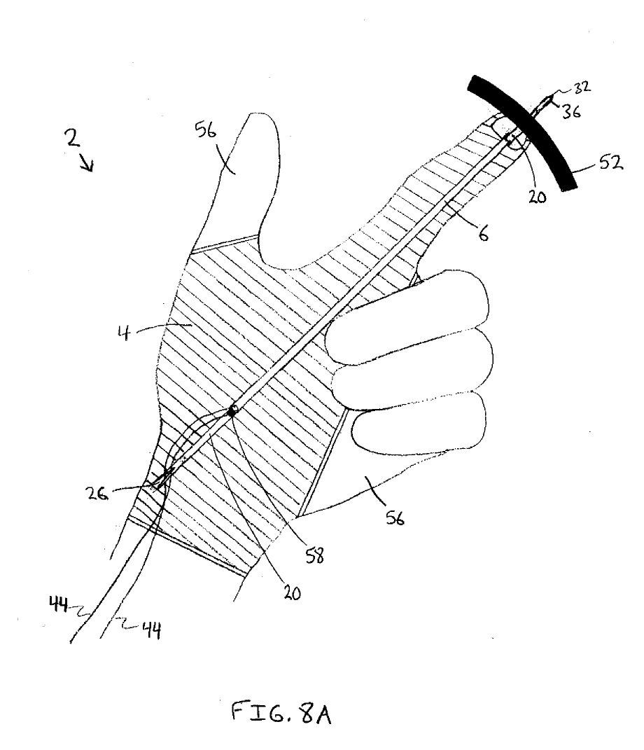

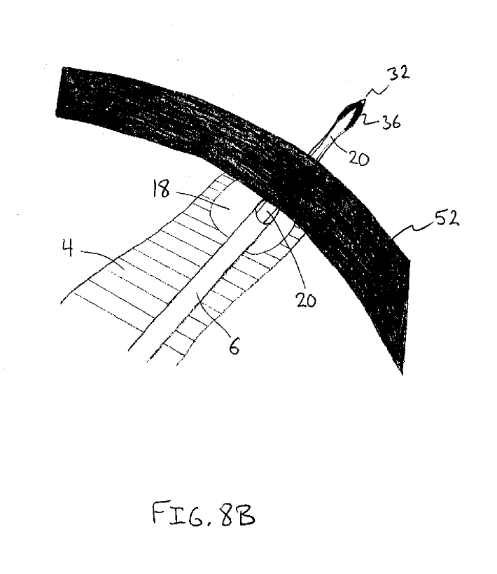

A depth marker 58 may be provided along the shaft of the insertion rod 20 to indicate to the operator when the distal tip of the insertion rod 20 has been advanced to a desired depth, as seen in FIGS. 6-7. The depth marker 58 may comprise any means suitable for indicating the depth of the insertion rod, such as printed markings or notches, and is preferably disposed closer to proximal end 22 than to distal end 24. The depth marker 58 will arrive at the proximal end 8 of the conduit 6 when the desired depth of penetration has been reached, as seen in FIGS. 7-8A.

As seen in FIGS. 2-3 and 5, the distal end 24 of the insertion rod 20 is fashioned to translocate the tissue anchor 50 through the conduit 6, and through a thickness of host tissue. Distal end 24 comprises a penetrating tip 32 that is configured to penetrate the target tissue. In this fashion, distal end 24 may be formed in the shape of a needle tip. The insertion rod 20 is designed to translocate the tissue anchor 50 in only one direction as it is advanced forward through the conduit 6 and host tissues. The insertion rod 20 is also designed to separate from the tissue anchor 50 as the insertion rod 20 is withdrawn from the host tissue, leaving the tissue anchor 50 within the host tissue.

The distal end 24 of the insertion rod 20 may contain lateral grooves 28 of a depth and width adequate to accept the frame 36 of the anchor element 50. These lateral grooves 28 are preferably in continuity with a distal groove 30 within the penetrating tip 32 of the distal end 24 of the insertion rod 20. The groove 30 in the penetrating surface of the insertion rod 20 is of a depth and width adequate to accept the leading end 38 of the anchor frame 36. In FIG. 3, line 31 illustrates one example of how deep groove 30. It is contemplated that different groove depths are within the scope of the present invention.

In a preferred embodiment, there is a recess 54 in the material of the insertion rod 20 that is disposed proximal to the lateral grooves 28. This recess 54 accommodates the lagging end 40 of the anchor frame 36. Recess 54 and the grooves 28 and 30 described above serve to reduce the thickness of the insertion rod/anchor assembly. In addition, the proximal recess 54 allows the lagging end 40 of the anchor element 50 to be dislodged from the insertion rod 20 by the surrounding host tissues as the rod 20 is withdrawn from the host tissues.

In a preferred embodiment shown in FIG. 4, the tissue anchor element 50 comprises a frame 36, a biocompatible fabric 42, and suture elements 44. The frame 36 is preferably composed of a strong flexible material that possesses the property of memory. Possible materials include, but are not limited to, heat treated stainless steal (memory wire), various metallic alloys, resins, plastics, silicon, synthetic rubbers, various bio-absorbable materials such as polyglycolic acid (vicryl), or some combination of these or any other suitable materials. The physical properties of the frame material allow the frame 36 to be compressed into an elongated shape as it is loaded onto the insertion rod 20 and advanced through the conduit and host tissues. The physical property of memory will cause the anchor frame 36 to expand once it is advanced completely through conduit and into sufficiently compliant tissue, such as adipose tissue.

In a preferred embodiment, such as shown in FIG. 4, the leading end 38 of frame 36 is substantially angled, while the lagging end 40 is substantially rounded. However, it is contemplated that a variety of different frame shapes are within the scope of the present invention.

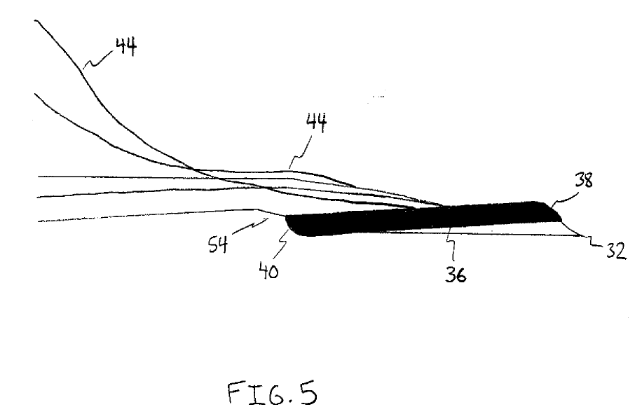

The biocompatible fabric element 42 of the anchor 50 preferably extends across a majority of the interior of anchor frame 36. A window 48 may be disposed in the fabric 42 at the leading end 38 of the anchor element 50. This window 48 is configured to receive the penetrating tip 32 of the insertion rod 20 as the anchor 50 is loaded onto the distal end 24 of the insertion rod 20, as seen in FIG. 5.

The biocompatible fabric 42 may comprise any material compatible with the human body. For example, fabric 42 may be composed of a permanent synthetic material, such a woven, knitted, or molded polypropylene. The fabric 42 may also be a biological allograft or xenograft. The fabric material may also include a bio-absorbable material, such as polyglycolic acid. The fabric 42 may be composed of any other biocompatible material having the softness, strength, and flexibility suitable for insertion, deployment, and retention within a body.

The fabric 42 can be attached to the anchor frame 36 by a variety of techniques including, but not limited to, adhesive or heat bonding. If the frame 36 and the fabric 42 are composed of the same material, they may be manufactured as a single piece, thereby circumventing the need to attach two separate elements. The fabric 42 may also have the property of memory, such that it will contribute to the expansive force of the frame 36 as it resumes it's original shape once it is deployed.

In a preferred embodiment, the suture element 44 is attached to the central region 46, or a location proximate the central region 46, of the biocompatible fabric 42. In another embodiment, a single strand of suture is looped through the biocompatible fabric in such a way that it can slide through the fabric if traction is applied to one arm of the suture. This later embodiment would allow the suture to be used as a pulley apparatus to elevate a tissue graft secured to the second arm of the looped suture strand.

The device of the present invention can be used for a variety of procedures requiring fixation of suture material at precise locations within a body. Among these procedures are vaginal reconstructive procedures including, but not limited to, vaginal vault suspension, uterine suspension, rectocele repair, cystocele repair, and urethral sling procedures. This apparatus can be used in the context of graft augmented procedures, or procedures in which the sutures are attached to the patient's own tissues.

The following describes the application of the present invention in the context of graft augmented vaginal vault suspension and rectocele repair. With adequate anesthesia established, and with the patient prepped and draped in a lithotomy position, the posterior vaginal wall is incised along the midline for a distance of approximately 5 cm. The posterior vaginal wall epithelium is dissected off the underlying rectum laterally to the pelvic floor musculature, and cephalad up to the ishial spines. The dissection is continued cephalad until the undersurface of the vaginal vault tibromuscularis is adequately exposed.

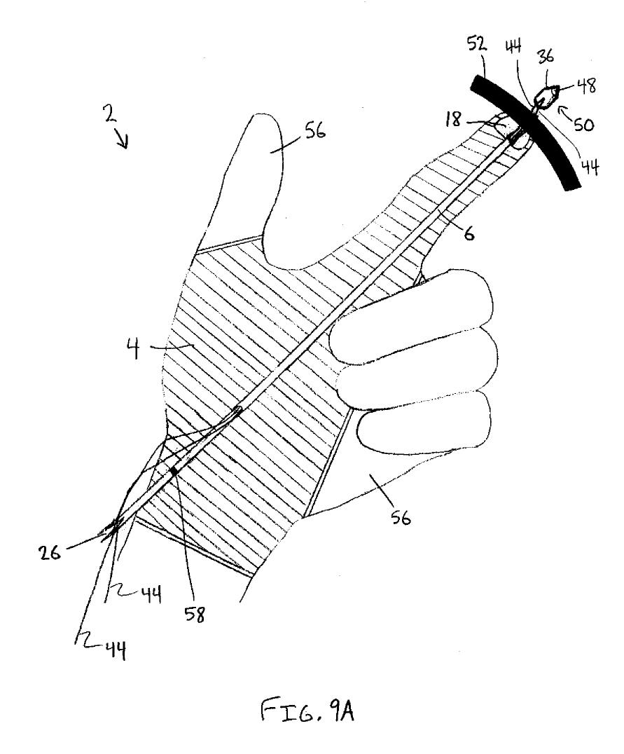

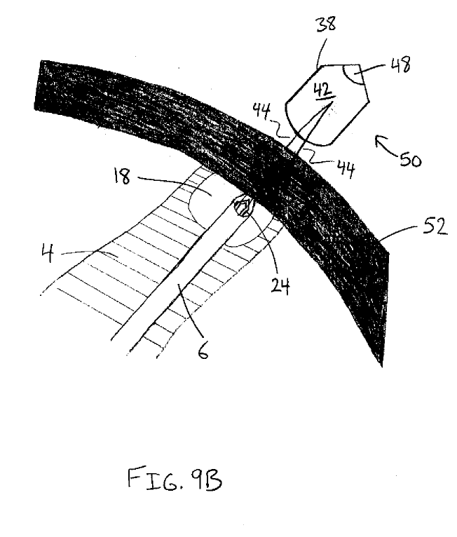

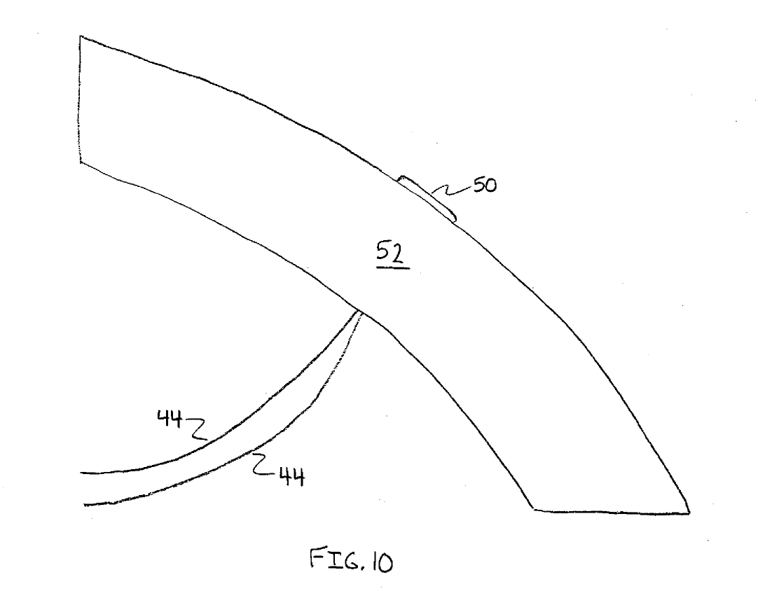

With the conduit 6 (either secured as a solo conduit directly on underglove 56 or grouped with conduit glove 4) being secured on and positioned by the index finger of the operator's left hand, the patient's left ishial spine is palpated with the ventral surface of the distal phalanx of the operators first finger. While the operator holds the tip of his first finger against this boney landmark, the tissue anchor/insertion rod assembly is inserted into the proximal end 8 of the conduit 6 and advanced through the conduit 6 until the tip 32 of the insertion rod 20 emerges from the distal end 10 of the conduit 14. The insertion rod is then advanced further pushing the tip of the insertion rod through the pelvic floor musculature just anterior to the ishial spine, as seen in FIGS. 8A-8B. The insertion rod is advanced until the depth marker 58 aligns with the proximal end 8 of the conduit 6, indicating that the lagging end 40 of the tissue anchor 50 is beyond the deep surface of the target tissue layer 52. The suture elements 44 are then dislodged from the holding slit 26 in the proximal end 22 of the insertion rod 20. The insertion rod 20 is then withdrawn from the conduit 6, leaving the tissue anchor 50 deployed within the host tissue 50, as seen in FIGS. 9A-10. The operator's hand is then removed, as the sutures 44 attached to the anchor 50 slide through the conduit 6.

Additional anchors can be placed at more distal locations along the left side of the pelvic floor. This procedure is repeated on the patient's opposite side using the operator's opposite hand in order to place anchors at corresponding symmetric positions along the pelvic floor. The sutures attached to the various anchors are then used to secure an approximately trapezoidal shaped piece of biological or synthetic graft material into position over the rectum and beneath the fibromuscularis of the vaginal vault. Excess suture is trimmed. A tacking suture is placed between the graft and the undersurface of the fibromuscularis of the vagina vault and the midline of the upper edge of the graft. The posterior vaginal wall epithelium is closed in the usual fashion.

It should be appreciated that the material used to fabricate the insertion rod is sufficiently flexible to allow the tip of the insertion rod to be deflected off of a boney structure as the insertion rod and anchor are advanced through tissues that are closely applied to bone. Additionally, the penetrating tip of the insertion rod may be slightly blunt so as to prevent the tip from imbedding into the periostium of a boney structure it may contact in the course of anchor insertion.

A similar technique can be used to perform a graft augmented cystocele repair.

The apparatus can be used to place tissue anchors through the pelvic floor musculature at the location appropriate for fixation of a mid-urethral sling. With anchors placed symmetrically in such a position, a small strip of graft material can be secured at the level of the midurethra to treat urinary incontinence. The flexible properties of the conduit and the insertion rod will allow the tip of the rod to be directed in a desired, relatively lateral, direction as the host tissue is penetrated by the tip of the insertion rod.

The apparatus can be used to perform a so-called “male sling” procedure by obtaining fixation points along the pelvic floor musculature at a suitable location for placement of a graft over the proximal urethra.

The tissue anchor-insertion rod assembly can also be used for laparoscopic procedures requiring fixation of suture material at precise locations within the host tissues. Examples of such procedures include laparoscopic uterine suspension or vagina vault suspension.

While embodiments and applications of this disclosure have been shown and described, it would be apparent to those skilled in the art that many more modifications and improvements than mentioned above are possible without departing from the inventive concepts herein. The disclosure, therefore, is not to be restricted except in the spirit of the appended claims.

What is claimed is:

1. A surgical implantation device comprising:
a flexible conduit having a proximal end and a distal end opposite said proximal end, said flexible conduit comprising an opening at said proximal end in communication with an opening at said distal end;
a means for securing said flexible conduit to an operator's hand over a surgical glove such that the position of said conduit may be adjusted by the movement of one of the operator's fingers;
a tissue anchor configured to enter said opening in said proximal end, be advanced through said conduit, and exit said opening in said distal end where it can be deployed beyond the surface of a target tissue layer;
a suture element connected to said tissue anchor, said suture element configured to extend from said tissue anchor through said conduit, from said distal end to said proximal end; and
a flexible rod having a proximal end and a distal end opposite said proximal end, said distal end of said rod configured to advance said tissue anchor through said conduit from said opening in said proximal end of said conduit to said opening in said distal end of said conduit and deploy said tissue anchor beyond the surface of said target tissue layer.

2. The device of claim 1, wherein said means for securing comprises a glove garment configured to be worn over the operator's hand, wherein said conduit is attached to said glove garment such that said conduit extends over the distal phalanx of either the operator's index finger or middle finger when said glove garment is worn over the operator's hand.

3. The device of claim 2, wherein said conduit is configured to extend from the palm region of the operator's hand to the distal phalanx of either the operator's index finger or middle finger when said glove garment is worn over the operator's hand.

4. The device of claim 3, wherein said glove garment is configured to:
cover at least a portion of the operator's palm;
wrap around the back of the operator's hand to securely hold itself in position on the operator's hand; and
extend over a substantial portion of at least one of the operator's index finger and middle finger.

5. The device of claim 3, wherein said glove garment is configured to:
cover a majority of the operator's palm region; and
completely envelope at least one of the operator's index finger and middle finger, wherein said glove garment comprises an opening configured to expose the ventral surface of operator's finger at the distal phalanx.

6. The device of claim 1, wherein said means for securing comprises an adhesive disposed along said conduit, said adhesive configured to secure said conduit to said surgical glove.

7. The device of claim 1, wherein said distal end of said rod is formed in the shape of a needle tip.

8. The device of claim 1, wherein said distal end of said rod is configured to:
hold said tissue anchor as said distal end of said rod is advanced through said conduit, to said distal end of said conduit, and through said target tissue layer; and
release said tissue anchor as said distal end of said rod is removed from said target tissue layer and said conduit.

9. The device of claim 8, wherein said tissue anchor comprises:
a frame forming an interior and having a leading end, a lagging end opposite said leading end;
a biocompatible fabric extending across said interior from one lateral side of said frame to the opposite lateral side; and
a window opening formed in between said leading end of said frame and said biocompatible fabric, wherein said window opening is configured to receive said distal end of said rod.

10. The device of claim 9, wherein said distal end of said rod comprises a groove configured to receive said frame of said tissue anchor.

11. The device of claim 9, wherein said suture element is attached to said biocompatible fabric at a position proximate the central region of said interior.

12. The device of claim 9, wherein said frame of said tissue anchor is formed from a strong flexible material having structural memory such that:
said frame may be compressed as it is loaded onto said rod and advanced through said conduit and said target tissue layer; and
said frame expands once it is advanced completely through said conduit and said target tissue layer.

13. The device of claim 10, wherein said rod comprises a recess disposed proximate said groove, said recess is configured to receive said lagging end of said frame.

14. The device of claim 13, wherein said groove extends from said recess, along one lateral side of said rod, around said distal end of said rod, along the opposite lateral side of said rod, back to said recess.

15. The device of claim 9, wherein said lagging end of said frame is substantially angled.

16. The device of claim 1, wherein said conduit is configured to extend from the palm region of the operator's hand to the distal phalanx of either the operator's index finger or middle finger when said conduit is secured to the operator's hand.

17. The device of claim 1, wherein said proximal end of said rod comprises a slit for receiving said suture element in such a way that said suture element is held securely in place within said slit.

18. A method for using a surgical implantation device comprising:
an operator securing a flexible conduit to an operator's hand over a surgical glove such that the position of said conduit may be adjusted by the movement of one of the operator's fingers, said conduit having a proximal end, a distal end opposite said proximal end, and an opening at said proximal end in communication with an opening at said distal end;
said operator positioning said distal end of said conduit proximate a target tissue layer by moving one of said fingers;
said operator advancing a tissue anchor through said opening at said proximal end, through said conduit, to and out of said opening at said distal end, and through said target tissue layer using a flexible rod, said rod having a proximal end and a distal end opposite said proximal end, wherein a suture element is connected to said tissue anchor and extends through said target tissue layer and through said conduit to said opening at said proximal end,
wherein said distal end of said rod holds said tissue anchor as said distal end of said rod is advanced through said conduit, to said distal end of said conduit, and through said target tissue layer, and said distal end of said rod releases said tissue anchor as said distal end of said rod is removed from said target tissue layer and said conduit.

19. The method of claim 18, wherein said conduit extends from the palm region of the operator's hand to the distal phalanx of either the operator's index finger or middle finger.

20. The method of claim 18, wherein said distal end of said rod is formed in the shape of a needle tip.
